(19) United States
(12) Plant Patent Application Publication
(10) Pub. No.: US 2017/0071115 P1
(43) Pub. Date: Mar. 9, 2017

(54) Bidens plant named 'KOIBID1346'

(50) ;

(71) Applicant: Koichiro Nishikawa, Nagi (JP)

(72) Inventor: Koichiro Nishikawa, Nagi (JP)

(21) Appl. No.: 14/756,434

(22) Filed: Sep. 4, 2015

(51) Int. Cl. A01H 5/00

(52) U.S. Cl. PLT/410

(57) ABSTRACT

A new and distinct cultivar of Bidens plant named ‘Koibid1346’, characterized by its compact, upright to somewhat outwardly spreading and mounding plant habit; vigorous growth habit; freely branching habit; early and freely flowering habit; long flowering period; and single-type inflorescences with greyed orange-colored ray florets and yellow-colored disc florets.

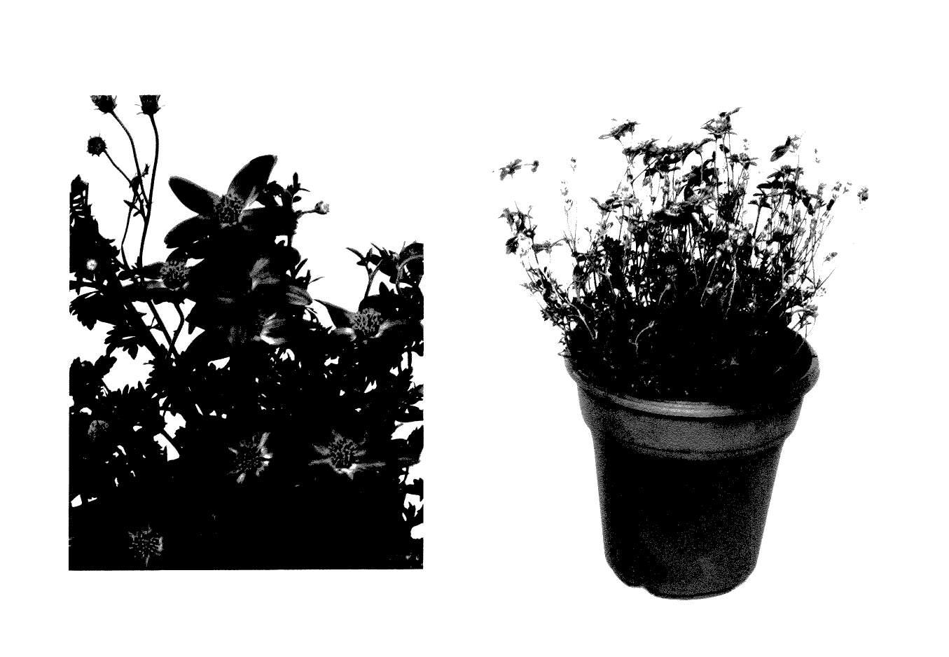

Botanical designation: Bidens triplinervia.

Cultivar denomination: ‘KOIBID1346’.

### BACKGROUND OF THE INVENTION

The present invention relates to a new and distinct cultivar of Bidens plant, botanically known as Bidens triplinervia and hereinafter referred to by the name ‘Koibid1346’.

The new Bidens plant is a product of a planned breeding program conducted by the Inventor in Okayama-ken, Japan. The objective of the breeding program is to create new freely-branching Bidens plants with unique ray floret coloration.

The new Bidens plant originated from a self-pollination made by the Inventor in Okayama-ken, Japan in January, 2010 of Bidens triplinervia ‘K-SAIKA001’, not patented. The new Bidens plant was discovered and selected by the Inventor as a single flowering plant from within the progeny of the stated self-pollination in a controlled greenhouse environment in Okayama-ken, Japan in May, 2010.

Asexual reproduction of the new Bidens plant by vegetative cuttings in a controlled environment in Okayama-ken, Japan since June, 2010 has shown that the unique features of this new Bidens plant are stable and reproduced true to type in successive generations.

### SUMMARY OF THE INVENTION

Plants of the new Bidens have not been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Koibid1346’. These characteristics in combination distinguish ‘Koibid1346’ as a new and distinct Bidens plant:

1. Compact, upright to somewhat outwardly spreading and mounding plant habit.

2. Vigorous growth habit.

3. Freely branching habit.

4. Early and freely flowering habit.

5. Long flowering period.

6. Single-type inflorescences with greyed orange-colored ray florets and yellow-colored disc florets.

Plants of the new Bidens differ primarily from plants of the parent, ‘K-SAIKA001’ in ray floret color as inflorescences of ‘K-SAIKA001’ have subdued orange and yellow bi-colored ray florets.

Plants of the new Bidens can be compared to plants of Bidens triplinervia ‘Haflpare’, disclosed in U.S. Plant patent application Ser. No. 14/544,724. In side-by-side comparisons conducted in Okayama-ken, Japan, plants of the new Bidens differed primarily from plants of ‘Haflpare’ in ray floret color as plants of ‘Haflpare’ have deep orange and yellow bi-colored ray florets.

Plants of the new Bidens can be compared to plants of Bidens triplinervia ‘Haflrest’, disclosed in U.S. Plant patent application Ser. No. 14/544,725. In side-by-side comparisons conducted in Okayama-ken, Japan, plants of the new Bidens differed primarily from plants of ‘Haflrest’ in ray floret color as plants of ‘Haflrest’ have dark orange-colored ray florets with yellow-colored margins.

Plants of the new Bidens can be compared to plants of Bidens triplinervia ‘Hafltibr’, disclosed in U.S. Plant patent application Ser. No. 14/544,732. In side-by-side comparisons conducted in Okayama-ken, Japan, plants of the new Bidens differed primarily from plants of ‘Hafltibr’ in ray floret color as plants of ‘Hafltibr’ have orange-colored ray florets with orange red-colored stripes.

Plants of the new Bidens can be compared to plants of Bidens triplinervia ‘Hafltifrpu’, disclosed in U.S. Plant patent application Ser. No. 14/544,730. In side-by-side comparisons conducted in Okayama-ken, Japan, plants of the new Bidens differed primarily from plants of ‘Hafltifrpu’ in ray floret color as plants of ‘Hafltifrpu’ have orange and yellow bi-colored ray florets.

Plants of the new Bidens can be compared to plants of Bidens triplinervia ‘Hafltufr’, disclosed in U.S. Plant patent application Ser. No. 14/544,729. In side-by-side comparisons conducted in Okayama-ken, Japan, plants of the new Bidens differed primarily from plants of ‘Hafltufr’ in ray floret color as plants of ‘Hafltufr’ have dark orange-colored ray florets with yellow to light yellow-colored apices.

Plants of the new Bidens can be compared to plants of Bidens triplinervia ‘Florbikano’, disclosed in U.S. Plant Pat. No. 25,087. In side-by-side comparisons conducted in Okayama-ken, Japan, plants of the new Bidens differed primarily from plants of ‘Florbikano’ in ray floret color as plants of ‘Florbikano’ have dark orange and yellow orange bi-colored ray florets.

Plants of the new Bidens can also be compared to plants of Bidens triplinervia ‘Yellow Charm’, not patented. In side-by-side comparisons conducted in Okayama-ken, Japan, plants of the new Bidens differed primarily from plants of ‘Yellow Charm’ in the following characteristics:
1. Plants of the new Bidens were larger than plants of ‘Yellow Charm’.
2. Plants of the new Bidens were more outwardly spreading and mounding than and not as upright as plants of ‘Yellow Charm’.
3. Plants of the new Bidens had narrower and darker green-colored leaves than plants of ‘Yellow Charm’.
4. Plants of the new Bidens and ‘Yellow Charm’ differed in ray floret color as plants of ‘Yellow Charm’ had solid yellow-colored ray florets.
5. Plants of the new Bidens were more high temperature-tolerant than plants of ‘Yellow Charm’.

### BRIEF DESCRIPTION OF THE PHOTOGRAPHS

The accompanying photographs illustrate the overall appearance of the new Bidens plant showing the colors as true as it is reasonably possible to obtain in colored reproductions of this type. Colors in the photographs may differ slightly from the color values cited in the detailed botanical description which accurately describe the colors of the new Bidens plant. The photograph on the right side of the sheet is a side perspective view of a typical flowering plant of ‘Koibid1346’ grown in a container. The photograph on the left side of the sheet is a close-up view of a typical flowering plant of ‘Koibid1346’.

### DETAILED BOTANICAL DESCRIPTION

The aforementioned photographs and following observations and measurements describe plants grown in 10-cm containers during the summer in a polyethylene-covered greenhouse in Bonsall, Calif. and under cultural practices which approximate those generally used in commercial Bidens production. During the production of the plants, day temperatures averaged 27° C., night temperatures averaged 18° C. and light levels ranged from 7,000 to 10,000 foot-candles. Plants were pinched one time and were six weeks old when the photographs and description were taken. In the following description, color references are made to The Royal Horticultural Society Colour Chart, 2007 Edition, except where general terms of ordinary dictionary significance are used.
Botanical classification: Bidens triplinervia ‘Koibid1346’.
Parentage: Self-pollination of Bidens triplinervia ‘K-SAIKA001’, not patented.
Propagation:
   Type.—By vegetative tip cuttings.
   Time to initiate roots, summer.—About one week at temperatures about 23° C.
   Time to initiate roots, winter.—About two weeks at temperatures about 23° C.
   Time to produce a rooted young plant, summer.—About three to four weeks at temperatures about 23° C.
   Time to produce a rooted young plant, winter.—About four to five weeks at temperatures about 10° C. to 15° C.
   Root description.—Fine, fibrous; white, close to 155D in color, actual color of the roots is dependent on substrate composition, water quality, fertilizer type and formulation, substrate temperature and physiological age of roots.
   Rooting habit.—Freely branching; dense.
Plant description:
   Plant and growth habit.—Compact, upright to somewhat outwardly spreading and mounding plant habit; vigorous growth habit and rapid growth rate.
   Branching habit.—Freely branching habit with about 38 lateral branches developing per plant; dense and bushy appearance.
   Plant height.—About 17.5 cm.
   Plant diameter or spread.—About 23 cm.
   Lateral branches.—Length: About 21 cm. Diameter: About 1.5 mm. Internode length: About 2.2 cm. Strength: Strong, flexible and wiry. Aspect: Upright to outwardly. Texture: Pubescent, incanous. Color, developing: Close to 144D. Color, developed: Close to N186C.
Leaf description:
   Arrangement.—Opposite, simple with deep convergent sinuses.
   Length.—About 1.7 cm.
   Width.—About 1.8 cm.
   Shape.—Leaf lobes are lanceolate.
   Apex.—Acute.
   Base.—Acute.
   Margin.—Deeply incised; crenate.
   Texture, upper and lower surfaces.—Smooth, mostly glabrous with scattered pubescence.
   Venation pattern.—Pinnate.
   Color.—Developing leaves, upper surface: Close to 137C. Developing leaves, lower surface: Close to 147B. Fully expanded leaves, upper surface: Close to N137A; venation, close to 146C. Fully expanded leaves, lower surface: Close to 147B often tinted with close to N186C; venation, close to 146C.
   Petioles.—Length: About 6 mm. Diameter: About 1 mm. Strength: Strong, flexible. Texture, upper and lower surfaces: Pubescence, minute. Color, upper and lower surfaces: Close to N79A.
Inflorescence description:
   Appearance.—Single type (daisy) solitary inflorescences developing on terminal and axillary peduncles; inflorescence form with ray and disc florets; inflorescences positioned on moderately strong peduncles; inflorescences face mostly upright to outwardly.
   Flowering habit.—Freely flowering habit with about 60 inflorescences and inflorescence buds at one time.
   Fragrance.—Faintly fragrant, pleasant.
   Flowering response.—Early flowering habit, plants begin flowering about four weeks after planting.
   Natural flowering season.—Long flowering period, plants flower continuously from spring until the autumn in temperate regions and year-round in milder climates.
   Inflorescence longevity.—Inflorescences last about five to seven days on the plant; inflorescences persistent.
   Inflorescence buds.—Height: About 8 mm. Diameter: About 6 mm. Shape: Nearly spherical. Texture: Slightly pubescent. Color: Close to 146A.
   Inflorescence size.—Overall diameter: About 3.5 cm. Overall height: About 1 cm. Disc diameter: About 7 mm.
   Receptacles.—Receptacle diameter: About 5 mm. Receptacle height: About 5 mm. Receptacle shape: Small, cup-shaped. Receptacle color: Close to 146B.
   Ray florets.—Number of ray florets per inflorescence: About seven to eight arranged in a single whorl. Length: About 1.7 cm. Width: About 7 mm. Shape: Elliptical. Apex: Emarginate. Base: Attenuate. Margin: Entire. Aspect: Mostly horizontal, reflexing with development. Texture, upper and lower surfaces: Longitudinally ribbed, glabrous. Luster, upper and lower surfaces: Matte. Color: When opening, upper surface: Close to 175B to 175C. When opening, lower surface: Close to 199B; towards the apex, close to 175D; towards the base, close to 7B. Fully opened, upper surface: Ground color, close to 7A overlain with close to 175B to 175C; towards the base, close to 7A tinted with close to N167C; color does not fade with development. Fully opened, lower surface: Ground color, close to 7B overlain with close to 174A; towards the base, close to 7B; color does not fade with development.
   Disc florets.—Number of disc florets per inflorescence: About 70 massed at the center of the receptacle. Length: About 8 mm. Diameter: About 1 mm. Shape: Tubular; apex, five-pointed. Color, when opening: Apex: Close to 160A. Mid-section: Close to 197B. Base: Close to 195D. Color, fully opened: Apex: Close to 160A. Mid-section: Close to 160D. Base: Close to 154D.
   Phyllaries.—Quantity per inflorescence: About 20 arranged in about three whorls. Length: About 4 mm. Width: Less than 1 mm. Shape: Lanceolate. Apex: Acute. Base: Truncate. Margin: Entire. Aspect: Slightly reflexed. Texture, upper and lower surfaces: Pubescent, minute hairs. Color, upper and lower surfaces: Close to 146A.
   Peduncles.—Length, terminal peduncle: About 3.8 cm. Length, first axillary peduncle: About 4.2 cm. Length, second axillary peduncle: About 5.4 cm. Diameter: About 1 mm. Strength: Moderately strong; flexible. Aspect: Terminal peduncles, mostly erect; axillary peduncles, about 15° to 20° from vertical. Texture: Pubescent, minute hairs. Color: Close to 148A.
   Reproductive organs.—Present on disc florets only; ray florets without visible reproductive organs. Androecium: Quantity per disc floret: Five. Filament length: About 2.5 mm. Filament color: Close to 1C. Anther shape: Lanceolate. Anther length: About 2 mm. Anther color: Close to N199B. Pollen amount: Scarce. Pollen color: Close to 7A. Gynoecium: Pistil length: About 8 mm. Style length: About 5 mm. Style color: Close to 3B. Stigma shape: Bi-parted. Stigma color: Close to 3A. Ovary color: Close to 144D.
   Seeds and fruits.—Seed and fruit development have not been observed on plants of the new Bidens.
Disease & pest resistance: Plants of the new Bidens have not been shown to be resistant to pathogens and pests common to Bidens plants.
Garden performance: Plants of the new Clematis have exhibited good tolerance to rain and wind and have been observed to tolerate temperatures from about 1° C. to about 45° C.

It is claimed:

1. A new and distinct Bidens plant named ‘Koibid1346’ as illustrated and described.
